(19) United States
(12) Patent Application Publication
(10) Pub. No.: US 2016/0015853 A1
(43) Pub. Date: Jan. 21, 2016

(54) Magnesium-calcium Silicate Bone Cement, Matrix Powder thereof and Producing Method Thereof

(71) Applicant: Chia-Tze Kao, (US)

(72) Inventors: Chia-Tze Kao, Taichung City (TW); Ming-You Shie, Kaohsiung City (TW)

(21) Appl. No.: 14/534,279

(22) Filed: Nov. 6, 2014

(30) Foreign Application Priority Data
Jul. 18, 2014 (TW) 103124673

(51) Int. Cl. A61L 24/02

(52) U.S. Cl. CPC A61L 24/02; A61L 2430/02

(57) ABSTRACT

The present invention provides a producing method for a degradable magnesium-calcium silicate bone cement. The invention solves the problems that the setting time is too long to cause bad mechanical property in conventional bone cements and also remains bioactivities and degradation ability. The invention has no cytotoxicity and enables to stimulate cells growth.

### BACKGROUND OF THE INVENTION

1. Field of Invention

This invention relates to bone cement for repairing bone defects, specifically degradable magnesium-calcium silicate bone cement. 2. Description of the Related Art

Calcium silicate-based bone cement is widely used in clinical situations. Silicon (Si) is an important trace element in an early stage of bone formation. Silicon (Si) is able to stimulate bone tissue regeneration and to increase bone cells proliferation. Calcium silicate-base bone cement becomes a major biomedical material in repairing or reconstructing bone defects. Materials such as mineral trioxide aggregate (MTA) and bio-glass are most common calcium silicate-based bone cements in the market. Clinical setting time of the mineral trioxide is around 162 minutes or more that leading to worse injectability and plasticity. The bio-glass has bad mechanical property that only suitable for repairing particular bone damage for which the bone doesn't need to support too much pressure, such like ear ossicle and finger bones.

Some solutions were revealed that trying to reduce the setting time of the conventional calcium silicate-based bone cement. These improved conventional calcium silicate-based bone cement may cause a worse mechanical property, degradation ability and plasticity thereof. Some macromolecular materials such as gelatin, chitosan or collagen are added into the conventional calcium silicate-based bone cement to improve the said mechanical property, degradation ability and plasticity. However, calcium silicate-bone cement with macromolecular materials may further increase setting time and worsen the bioactivity. Thus, the conventional calcium silicate-based bone cement has many aforementioned disadvantages that need to be solved.

### SUMMARY OF THE INVENTION

In order to solve the disadvantages and shortcomings of the conventional calcium silicate-based bone cement. The present invention provides a degradable magnesium-calcium silicate bone cement and a producing method thereof to obviate or mitigate the shortcoming of the prior art.

The producing method of a degradable magnesium-calcium silicate bone cement having steps of:
hydrolyzing a tetraethyl orthosilicate with a nitric acid;
mixing with a calcium nitrate and a magnesium nitrate;
reacting with an alcohol to obtain a mixture liquid;
drying the mixture liquid by a heating means;
sintering and graining the mixture liquid to obtain a magnesium-calcium silicate bone cement powder; and
blending a secondary water or a phosphate solution with the magnesium-calcium silicate bone cement powder uniformly to form the degradable magnesium-calcium silicate bone cement.

Thus, the present invention achieves advantages as below.

1. The present invention has no cytotoxicity and is able to stimulate cells to attach and grow.

2. The present invention provides improved bioactivity and degradation ability compared to the conventional calcium silicate-based bone cement.

3. The present invention provides great ability of cell differentiation.

### BRIEF DESCRIPTION OF THE DRAWINGS

FIG. 1 is an XRD pattern of each magnesium-calcium silicate bone cement testing block of the present invention;

FIG. 2 is surface SEM observation result for growths of apatite of the present invention after steeped in stimulated body fluid for 24 hours;

FIG. 3 illustrates mass loss of the present invention in different blending ration after immersed in stimulated body fluid for 24 hours;

FIG. 4 is a bar diagram of cells attachment and growth on the present invention;

FIG. 5 is surface SEM observation result for the primary human pulp fibroblast (HPF) attachment and growth on the present invention;

FIG. 6A is a test result for phosphatase secretion of the primary human pulp fibroblast (HPF) growth on present invention;

FIG. 6B illustrates phosphatase secretion and calcium deposition of the present invention;

FIG. 6C is a bar diagram of the primary human pulp fibroblast (HPF) attachment and growth on the present invention;

FIG. 7A illustrates angiogenesis of the human umibilical vein endothelial cell grow on present invention;

FIG. 7B is a bar diagram of vWF production of the human umibilical vein endothelial cell growth on present invention; and

FIG. 7C is a bar diagram of Ang-1 production of the human umibilical vein endothelial cell grown on present invention.

### DETAILED DESCRIPTION OF THE INVENTION

A producing method of degradable magnesium-calcium silicate bone cement in accordance with the present invention has processing steps comprising of:
hydrolyzing a tetraethyl orthosilicate (TEOS) with a nitric acid;
mixing with a calcium nitrate and a magnesium nitrate;
reacting with an alcohol 0.5˜1.5 hours to obtain a mixture liquid;
drying the mixture liquid;
sintering and graining the mixture liquid to obtain a magnesium-calcium silicate bone cement powder.

The concentration of the nitric acid is 0.5˜5N. The concentration of alcohol is 99.5%. The mixture liquid is dried at 40° C.˜100° C. for a day by a heating means, and then continually dried at 100° C.˜200° C. The heating means may be directly heating, infrared heating and thermal resistance heating means. The mixture liquid is continually sintered at temperature 500° C.˜1400° C. for 1˜12 hours. The mixture liquid is continually grained for 4˜18 hours by a miller.

Blending a secondary water or a phosphate solution with the magnesium-calcium silicate bone cement powder uniformly to form a degradable magnesium-calcium silicate bone cement. The phosphate solution may accelerate the setting time and the time of hydration reaction after blending the magnesium-calcium silicate bone cement with the water or the phosphate solution.

Different blending ratio of the secondary water or the phosphate solution to the magnesium-calcium silicate bone cement powder is shown in chart 1 as blow.

CHART 1

| | Ca:Si:Mg | L/P ratio | Setting time | DTS |
|---|---|---|---|---|
| Code | (mole ratio) | (mL/g) | (min) | (MPa) |
| Mg0 | 60:40:0 | 0.42 | 16.3 ± 1.3 ^{a} | 2.5 ± 0.12 ^{d} |
| Mg5 | 55:40:5 | 0.40 | 20.5 ± 1.4 ^{b} | 2.4 ± 0.11 ^{d} |
| Mg10 | 50:40:10 | 0.38 | 27.1 ± 1.8 ^{c} | 2.1 ± 0.12 ^{e} |

According to the above chart 1, physical properties and chemical properties of the degradable magnesium-calcium silicate bone cement in different blending ratio are as followings.

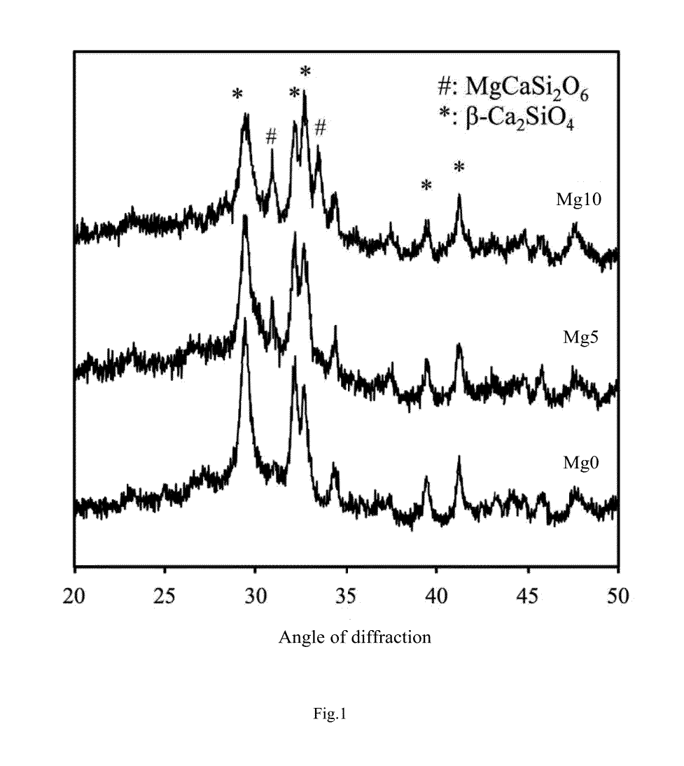

With reference to FIG. 1, XRD patterns showed main constituent of the degradable magnesium-calcium silicate bone cement is β-phase calcium silicate (β-Ca₂SiO₄). The testing method has steps of blending the secondary water or the phosphate solution with the magnesium-calcium silicate bone cement powder in different ratio to form the degradable magnesium-calcium silicate bone cement. The different blending ratio of the degradable magnesium-calcium silicate bone cement is filled into a mould to form a degradable magnesium-calcium silicate bone cement testing block and then preserved in a hydrate environment. Each mould used in the present embodiment has a diameter of 6 mm and a height of 3 mm. Temperature of the hydrate environment is controlled at 37° C. Relative humidity of the hydrate environment is 100%.

The degradable magnesium-calcium silicate bone cement testing block is removed from the mould after hydration reaction completed. With reference to chart 1, the setting time of the degradable magnesium-calcium silicate bone cement testing block in different blending ratio is measured. A testing standard of setting time used in the accordance with the present invention is ASTM C 187-98 (American Society for Testing and Materials).

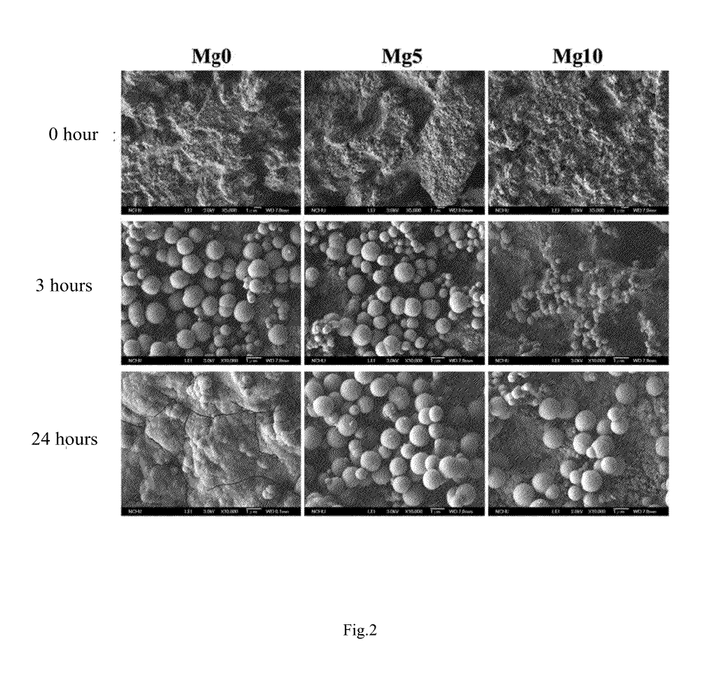

The degradable magnesium-calcium silicate bone cement testing block in different blending ratio is being immersed into a stimulated body fluid (SBF) 10 mL for a period of time and then dried by an oven. A growth of apatite on the degradable magnesium-calcium silicate bone cement testing block is observed by an electronic microscope. With reference to FIG. 2, growths of apatite of sample Mg0, the degradable magnesium-calcium silicate bone cement without containing magnesium ion, are plenty generated after immersing in SBF for 3 hours. Growths of apatite of sample Mg10, the degradable magnesium-calcium silicate bone cement containing 10% magnesium ion, are still plenty generated after immersing in SBP for a day. The growth of apatite in the above results means that the present invention has great bioactivity.

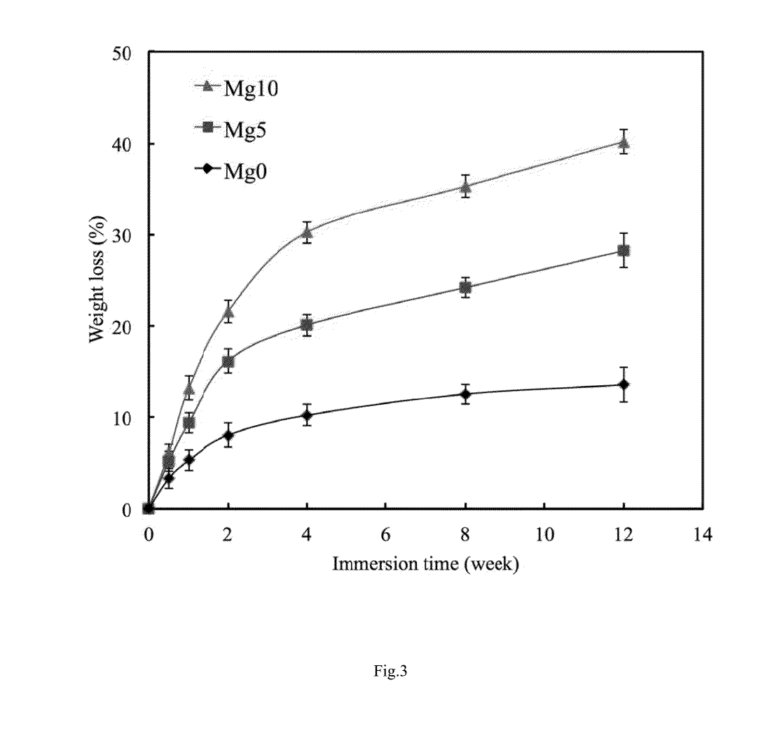

With reference to chart 1, the degradable magnesium-calcium silicate bone cement testing block in different blending ration is being immersed into the stimulated body fluid (SBF) 10 mL for a period of time and then dried by an oven. A mass loss of the magnesium-calcium silicate bone cement testing block and strength of surface structure of the magnesium-calcium silicate bone cement testing block are observed by an electronic microscope. With reference to FIG. 3, the mass loss of the magnesium-calcium silicate bone cement testing block is increased according to content of the magnesium ion in the magnesium-calcium silicate bone cement. The mass loss of sample Mg10 is around 40% after immersed in the SBP in 12 weeks.

The testing method of the present invention has no cytotoxicity and is able to stimulate cells to attach and grow may have steps comprising of:
sterilizing the magnesium-calcium silicate bone cement by a 75% ethanol solution;
exposing the magnesium-calcium silicate bone cement to a UV light for an hour; and
culturing primary human pulp fibroblast (HPF) on the sterilized magnesium-calcium silicate bone cement.

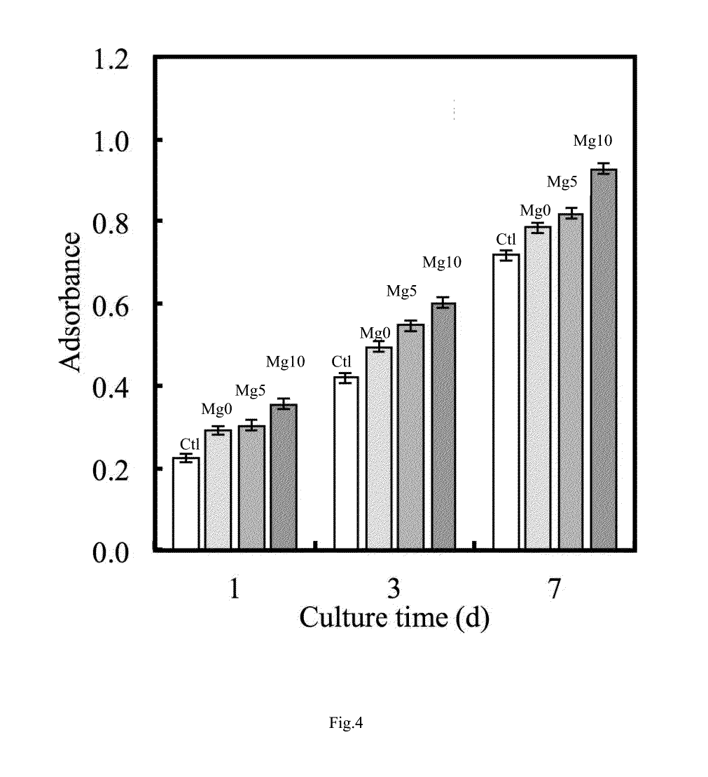

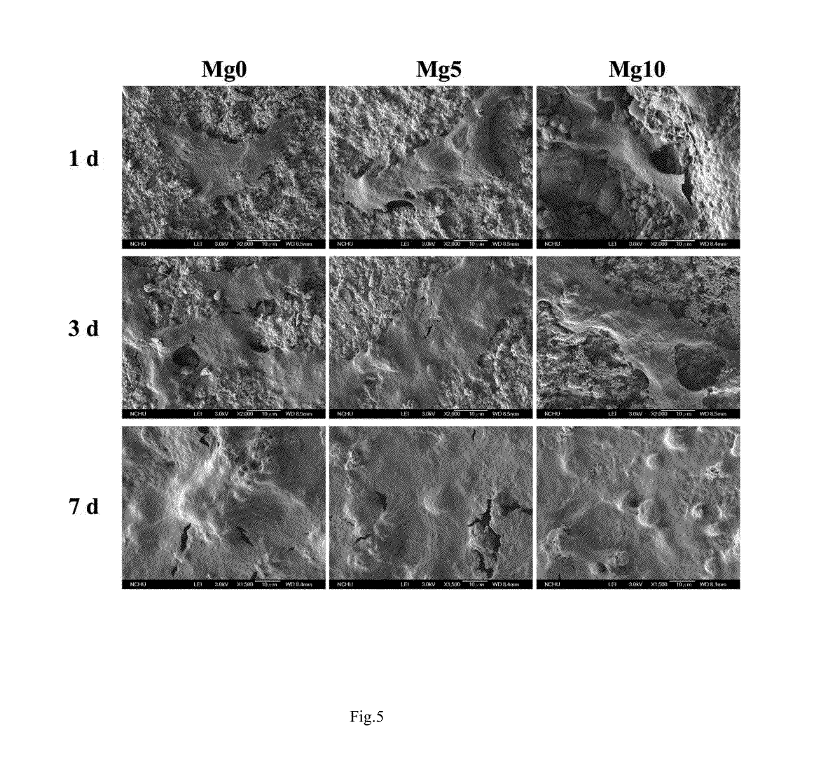

A growth rate of the primary human pulp fibroblast (HPF) is observed in different timing. With reference to FIG. 4, the growth rate of the primary human pulp fibroblast (HPF) is increased according to content of the magnesium ion in the degradable magnesium-calcium silicate bone cement. With reference to FIG. 5, the present invention shows more non-cytotoxicity and has better abilities to stimulate cells to attach and grow according to content of the magnesium ion in the degradable magnesium-calcium silicate bone cement.

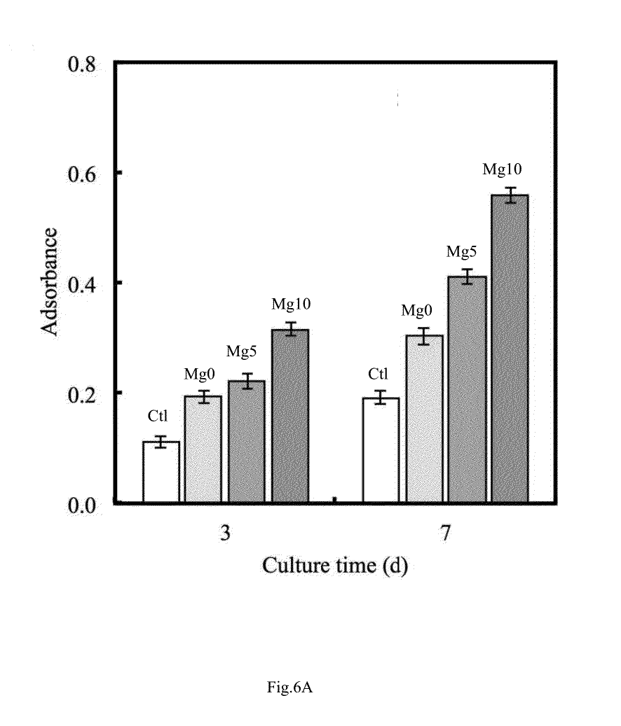

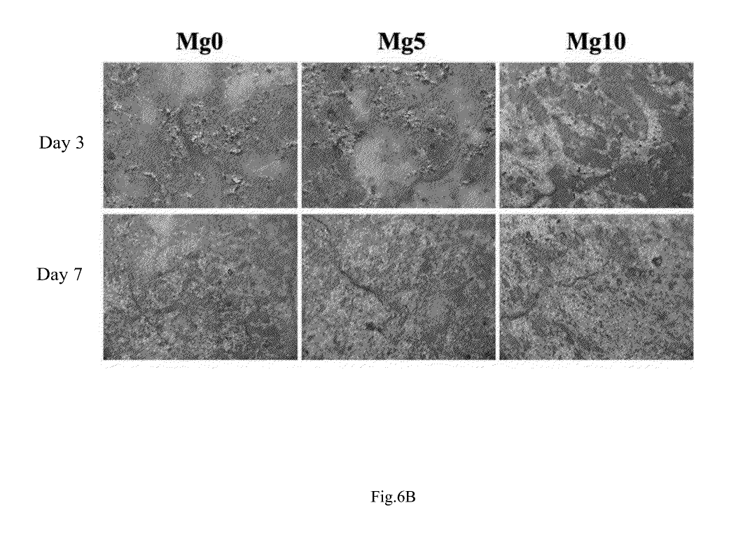

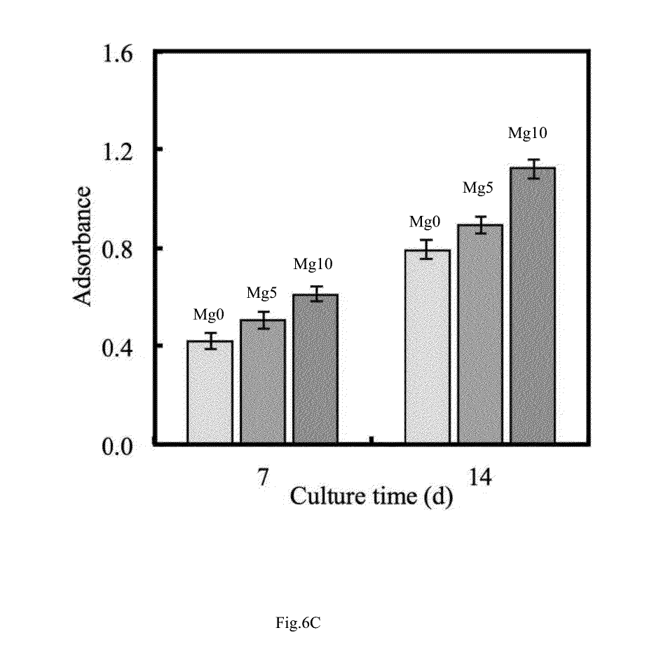

An activity of phosphatase and quantity of calcium deposition are an important index for bone cell differentiation. The primary human pulp fibroblast (HPF) is cultured on the sterilized magnesium-calcium silicate bone cement. The activity of phosphatase and the quantity of calcium deposition of the primary human pulp fibroblast (HPF) are observed in different time. With reference to FIG. 6A˜FIG. 6C, the activity of phosphatase and the quantity of calcium deposition of the primary human pulp fibroblast (HPF) are increased when the content of the magnesium ion in the magnesium-calcium silicate bone cement are increased, which means the present invention is able to stimulate cell differentiation.

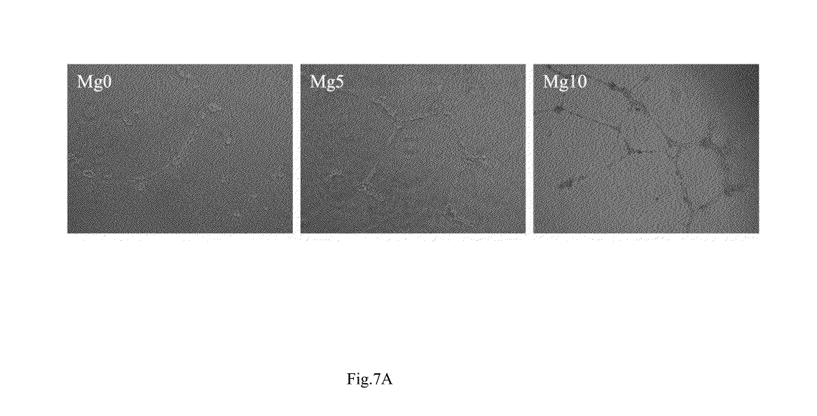

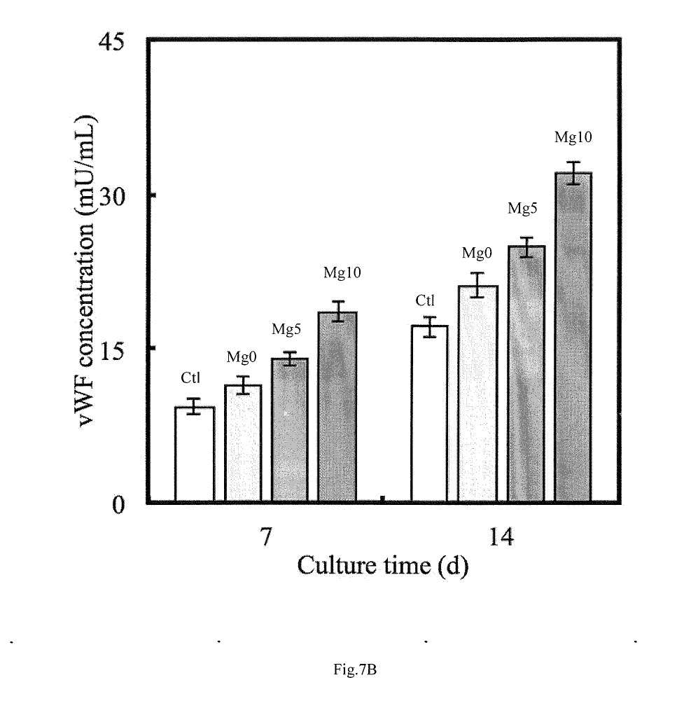

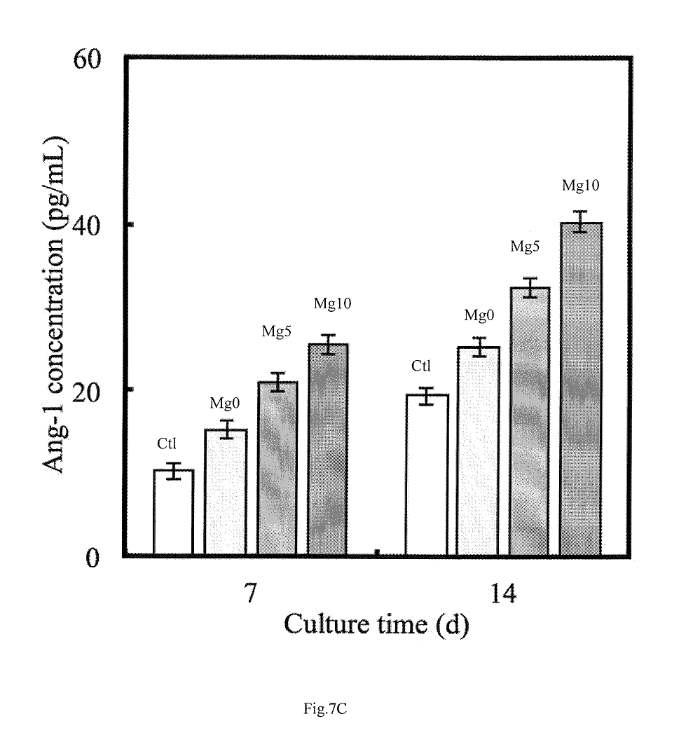

To test the present invention is able to promote angiogenesis of cells, human umibilical vein endothelial cell (HUVEC) is cultured on the sterilized magnesium-calcium silicate bone cement. With reference to FIG. 7A˜FIG. 7C, the present invention shows great ability of angiogenesis according to content of the magnesium ion increased in the magnesium-calcium silicate bone cement. The generation of angiogenic proteins like angiopoietin-1 (Ang-1) and von Willebrand factor (vWF) are also increased according to content of the magnesium ion in the magnesium-calcium silicate bone cement. Thus, the present invention achieves advantages as below. 1. The present invention has no cytotoxicity and is able to stimulate cells to attach and grow. 2. The present invention provides improved bioactivity and degradation ability compared to the conventional calcium silicate-based bone cement. 3. The present invention provides great ability of cell differentiation.

What is claimed is:

1. A producing method of a degradable magnesium-calcium silicate bone cement having steps of:
hydrolyzing a tetraethyl orthosilicate with a nitric acid;
mixing with a calcium nitrate and a magnesium nitrate;
reacting with an alcohol to obtain a mixture liquid;
drying the mixture liquid by a heating means;
sintering and graining the mixture liquid to obtain a magnesium-calcium silicate bone cement powder; and
blending a secondary water or a phosphate solution with the magnesium-calcium silicate bone cement powder uniformly to form the degradable magnesium-calcium silicate bone cement.

2. The producing method of a degradable magnesium-calcium silicate bone cement as claimed in claim 1, wherein
the degradable magnesium-calcium silicate bone cement is prepared by a sol-gel producing method;
the concentration of the nitric acid is 0.5 N˜5N;
the mixture liquid is obtained by reacting with the alcohol 0.5˜1.5 hours;
the heating means is directly heating, infrared heating or thermal resistance heating means;
the mixture liquid is continually sintered at temperature 500° C.˜1400° C. for 1˜12 hours; and
the mixture liquid is continually grained for 4˜18 hours by a miller.

3. A degradable magnesium-calcium silicate bone cement having a secondary water or a phosphate solution being uniformly blended with a magnesium-calcium silicate bone cement powder, wherein
a main constituent of the magnesium-calcium silicate bone cement powder in the degradable magnesium-calcium silicate bone cement is β-phase calcium silicate; and
range of the mole ratio of calcium, silicon and magnesium in magnesium-calcium silicate bone cement is from 60:40:0 to 50:40:10.

4. The degradable magnesium-calcium silicate bone cement as claimed in claim 3, the magnesium-calcium silicate bone cement powder is produced by a sol-gel method having steps of:
hydrolyzing a tetraethyl orthosilicate with a nitric acid;
mixing with a calcium nitrate and a magnesium nitrate;
reacting with an alcohol to obtain a mixture liquid;
drying the mixture by a heating means; and
sintering and graining the mixture liquid to obtain a magnesium-calcium silicate bone cement powder.

5. The degradable magnesium-calcium silicate bone cement as claimed in claim 4, wherein a blending ratio of the secondary water or the phosphate solution to the magnesium-calcium silicate bone cement powder is between 0.38 mL/g˜0.42 mL/g.

6. A matrix powder of a magnesium-calcium silicate bone cement is produced by a sol-gel method, wherein
a main constituent of the magnesium-calcium silicate bone cement is β-phase calcium silicate.

7. The matrix powder of the magnesium-calcium bone cement as claimed in claim 6, the sol-gel method having steps of:
hydrolyzing a tetraethyl orthosilicate with a nitric acid;
mixing with a calcium nitrate and a magnesium nitrate;
reacting with an alcohol to obtain a mixture liquid;
drying the mixture by a heating means; and
sintering and graining the mixture liquid to obtain a magnesium-calcium silicate bone cement powder.

8. The matrix powder of the magnesium-calcium bone cement as claimed in claim 7, wherein the mole ration of calcium, silicon and magnesium is ranging between 0.38 mL/g˜0.42 mL/g.
